(19) United States
(12) Patent Application Publication
(10) Pub. No.: US 2026/0053902 A1
(43) Pub. Date: Feb. 26, 2026

(54) COMPOSITIONS AND METHODS FOR REDUCING THE RATE OF TYPE 1 DIABETES

(71) Applicant: Wisconsin Alumni Research Foundation, Madison, WI (US)

(72) Inventor: Michael MacDonald, Madison, WI (US)

(73) Assignee: Wisconsin Alumni Research Foundation, Madison, WI (US)

(21) Appl. No.: 19/150,045

(22) PCT Filed: Jan. 25, 2024
(86) PCT No.: PCT/US2024/012892

§ 371 Date: Jul. 22, 2025

Related U.S. Application Data
(60) Provisional application No. 63/481,664, filed on Jan. 26, 2023.

(51) Int. Cl. A61K 39/00; A61K 31/353; A61K 36/63; A61K 38/00; A61P 3/10; A61P 37/04; C07K 14/47

(52) U.S. Cl. CPC A61K 39/0008; A61K 31/353; A61K 36/63; A61P 3/10; A61P 37/04; C07K 14/4713; A61K 38/00

(57) ABSTRACT

Described herein are methods of treating a human child including administering to the human child a compound selected from epigallocatechin gallate (EGCG), or a derivative thereof selected from epicatechin (EC), epigallocatechin (RGC) and epicatechin gallate (ECG), sulforaphane, olive oil, phylloquinone, quercetin, safranal, hydroxtyrosol, menaquinone-4, menaquinone-7, oleocanthal, peptide Ins-1 B9-23 19CAM22R-E, peptide Ins-1 B12-23-19CAM, peptide Ins-1 B12-23 19CAM22R-E, or a combination of any of the foregoing, wherein administration of the treatment is started between birth and six months of age. Also described are methods of treating a pregnant human or a nursing human mother who has recently given birth to an infant. Further described are insulin B chain mimotope peptides.

### CROSS-REFERENCE TO RELATED APPLICATIONS

This application claims priority to U.S. Provisional Application 63/481,664 filed on Jan. 26, 2023, which is incorporated herein by reference in its entirety.

### FIELD OF THE DISCLOSURE

The present disclosure is related to compositions and methods for the prevention and/or reducing the rate of type 1 diabetes, particularly treatments that can be started in young infants at risk of developing type I diabetes.

### BACKGROUND

Type 1 diabetes in children is a chronic condition in which the body no longer produces an important hormone (insulin) which is required by the body to survive. Type 1 diabetes in children used to be known as juvenile diabetes or insulin-dependent diabetes.

Type 1 diabetes is an autoimmune disorder in which the immune system attacks the cells in the pancreas that make insulin. Children with type 1 diabetes must have daily insulin to keep the blood glucose level within normal ranges. Insulin is injected by syringe or via an insulin pump. In addition, management of diet and exercise plus multiple checking of blood glucose are important for diabetes management. However, even with the advanced technology now available normal regulation of blood sugar levels is still imperfect and laborious for the patient and family. The lifestyle of the patient and family is still highly abnormal and stressful.

While methods of diabetes management are well-established, there are no established methods to prevent or reduce the rate of type 1 diabetes, particularly in children.

### BRIEF SUMMARY

In an aspect, a method of treating a human child comprises administering to the human child a compound selected from epigallocatechin gallate (EGCG), epicatechin (EC), epigallocatechin (RGC), epicatechin gallate (ECG), sulforaphane, olive oil, phylloquinone, quercetin, safranal, hydroxtyrosol, menaquinone-4, trans menaquinone-7, oleocanthal, peptide Ins-1 B9-23 19CAM22R-E, peptide Ins-1 B12-23-19CAM, peptide Ins-1 B12-23-19CAM22R-E, or a combination of any of the foregoing, wherein administration of the treatment is started between birth and six months of age.

In another aspect, a method of treating a pregnant human or a nursing human mother who has recently given birth to an infant, comprising administering to the pregnant human and/or to the nursing human mother a compound selected from epigallocatechin gallate (EGCG), epicatechin (EC), epigallocatechin (RGC), epicatechin gallate (ECG), sulforaphane, olive oil, phylloquinone, quercetin, safranal, hydroxtyrosol, menaquinone-4, trans menaquinone-7, oleocanthal, peptide Ins-1 B9-23 19CAM22R-E, peptide Ins-1 B12-23-19CAM, peptide Ins-1 B12-23-19CAM22R-Esafranalor a combination of any of the foregoing, wherein administration to the pregnant mother is begun during the final two months of gestation.

In yet another aspect, included is an insulin B chain mimotope peptide Ins-1 B9-23 19CAM22R-E having sequence SHLVEALYLVCGEEG (SEQ ID NO: 1) modified by treating the peptide with iodoacetamide to carboxyamidomethylate the cysteine residue, peptide Ins-1 B12-23-19CAM having sequence VEALYLVCGERG (SEQ ID NO: 2) modified at modified by treating the peptide with iodoacetamide to carboxyamidomethylate the cysteine residue, or peptide Ins-1 B12-23-19CAM22R-E having sequence VEALYLVCEGEEG (SEQ ID NO: 3) modified by treating the peptide with iodoacetamide to carboxyamidomethylate the cysteine residue.

### DETAILED DESCRIPTION

Described herein are chemical compounds and modified insulin peptides that have prevented type 1 diabetes in a mouse model of type 1 diabetes. The chemicals and modified peptides can be administered to mothers during pregnancy and while nursing as well as to infants and children, for example from the early days of life. Without being held to theory, it is believed that the compounds described herein induce immune tolerance to insulin and other antigens in type 1 diabetes and prevent or reduce the rate of type 1 diabetes.

Human infants develop immune tolerance by age 6 months. In their development between birth and 6 months, their immune systems are maturing and they make antibodies and antigenic cells to foreign proteins such as those from bacteria after 6. In utero, babies receive antibodies from their mothers, and right after birth they also receive antibodies from their mother's milk if the mother is nursing. Because of this, it is recommended that the treatments described herein be started as close to birth, particularly as the treatments are not harmful even to babies. As shown in the experiments described herein, administering some, but all, of the compounds described herein when started post-wean at 3 weeks of age decreased the rate of diabetes. Importantly, because some of the compounds that lowered the rate of diabetes when started early in life did not decrease the rate of diabetes in the mouse model when started later in life, it is important to start each of the compounds early in life. For comparison of the mouse studies to administration to humans, starting a treatment at age 5-6 weeks in a mouse is at an age where the developmental age of the mouse is relatively much older, for example, six weeks for a mouse is an age where they can start getting pregnant. At 30-35 days of age mice are sexually mature. In general, mice aged 3 to 6 months are equivalent to humans aged 20-30 years, and mice aged 1 and 2 months are equivalent to human aged 12.5-14 years and 16-20 human years. These comparisons are helpful when extending the mouse studies described herein to humans.

Previously, Fu et al. (“Epigallocatechin gallate delays the onset of type 1 diabetes in spontaneous non-obese diabetic mice”, Br J Nutr. 105(8), 1218-1225, (2011)) described a study in which a low concentration (1 mM) of EGCG in drinking water was asserted to delay the onset of type 1 diabetes in the NOD mouse. The inventors could not replicate the results of this study. Importantly, the Fu et al. study differed from the studies described herein in that the mice were not given EGCG until after age 5 weeks. Also the Fu et al. study was only carried out until the mice were 32 weeks old. At 32 weeks of age 25% of the 12 mice that were treated with EGCG had diabetes and 67% of the 12 control mice had diabetes. In four studies with EGCG described herein there were 26 to 37 mice per study group, providing greater statistical significance to the studies. A total of 118 mice received EGCG in these four studies (Table 1). Also, in the studies described herein, except for the one group of mice started on EGCG post wean at 21 days of age, all mice were started on EGCG shortly after birth at 2 to 5 days of age. Also importantly, in the studies described herein, nondiabetic mice were kept alive until they were 40 to 41 weeks old before they were euthanized. This is important because NOD mice can certainly develop diabetes after age 32 weeks. For example, in the mice treated with saline as a control there were two groups of 15 mice that had a rate of diabetes of 67% and 47%, respectively, at 32 weeks of age but by 40 weeks of age each of these two groups had a rate of diabetes equal to 80%. Only one group of 7 mice had a very high rate of diabetes (86%) at 32 weeks of age. (See controls in Table 1). Taken together, not only was the inventor unable to replicate the results of Fu et al., the inventor determined the importance of beginning EGCG administration in mice shortly after birth at 2 to 5 days of age, rather than the 5 weeks of Fu et al. In addition, carrying studies out to 40 weeks instead of 32 weeks can be important to determine if the compounds are effective in reducing the rate of diabetes in the mouse model. In addition to the four studies with EGCG described above, six additional studies were performed on about 137 mice that received EGCG. Results with these are described below.

Thus, in the methods described herein, administration of the treatment is started as soon as possible after birth well before six months of age. Without being held to theory, it is believed that such administration should be started prior to the development of immune tolerance in the infant.

In an aspect method of treating a human child comprises administering to the human child a compound selected from epigallocatechin gallate (EGCG), epicatechin (EC), epigallocatechin (RGC), epicatechin gallate (ECG), sulforaphane, olive oil, phylloquinone, quercetin, safranal, hydroxtyrosol, menaquinone-4, menaquinone-7, oleocanthal, peptide Ins-1 B9-23 19CAM22R-E, peptide Ins-1 B12-23-19CAM, peptide Ins-1 B12-23-19CAM22R-E, or a combination of any of the foregoing, wherein administration of the treatment is started as soon as possible after birth such as between birth and six months of age or older. In an aspect, the treatment may be started first to the pregnant mother of the infant and continued to the infant between birth and six months of age or older.

EGCG (epigallocatechin gallate; (2R,3R)-5,7-Dihydroxy-2-(3,4,5-trihydroxyphenyl)-3,4-dihydro-2H-1-benzopyran-3-yl 3,4,5-trihydroxybenzoate) has the structure

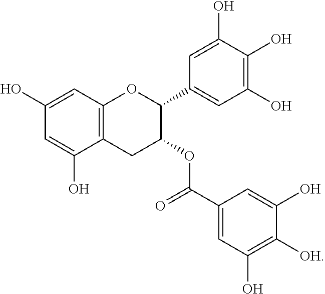 [SMILES: O=C(O[C@@H]1Cc2c(O)cc(O)cc2O[C@@H]1c1cc(O)c(O)c(O)c1)c1cc(O)c(O)c(O)c1]

EGCG is the most abundant catechin found in tea. Derivatives of EGCG include epicatechin (EC), epigallocatechin (RGC) and epicatechin gallate (ECG). In an aspect, pure EGCG, that is EGCG which has been separated from the non-EGCG components of a food source such as green tea, is used in the compositions and methods described herein. In an aspect, pure EC, that is EC which has been separated from the non-EC components of a food source sch as tea, berries or cocoa, is used in the compositions and methods described herein. In an aspect, pure RGC, that is RGC which has been separated from the non-RGC components of a food source such as green tea, is used in the compositions and methods described herein. In an aspect, pure ECG, that is ECG which has been separated from the non-ECG components of a food source such as green tea, is used in the compositions and methods described herein.

Sulforaphane (1-Isothiocyanato-4-(methanesulfinyl)butane) is an organosulfur compound found in cruciferous vegetables. In an aspect, pure sulforaphane, that is sulforaphane which has been separated from the non-sulforaphane components of a food source such as cruciferous vegetables, is used in the compositions and methods described herein.

Olive oil comprises triglyceride esters of oleic acid, and other fatty acids such as linoleic acid and palmitic acid. The most active diabetes preventive ingredients in olive oil are most likely polyphenols such as hydroxytyrosol, tyrosol, oleuropein, oleacein, oleocanthal, elenolic acid, alpha-tocopherol, 10-hydroxyoleurpein, ligstroside, 10-hydroxyligoside and flavonoids.

Phylloquinone is vitamin K1 a prenylated naphthoquinone. In an aspect, pure phylloquinone, that is phylloquinone which has been separated from the non-phylloquinone components of a food source such as green leafy vegetables and vegetable oil, is used in the compositions and methods described herein.

Quercetin (2-(3,4-Dihydroxyphenyl)-5,7-dihydroxy-4H-1-benzopyran-4-one) is a plant flavonol having the following structure

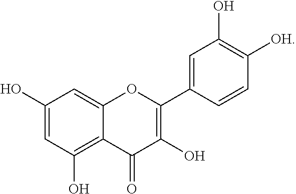 [SMILES: O=c1c(O)c(-c2ccc(O)c(O)c2)oc2cc(O)cc(O)c12]

In an aspect, pure quercetin, that is quercetin which has been separated from the non-quercetin components of a food source such as fruits and vegetables, is used in the compositions and methods described herein.

Safranal (2,6,6-Trimethylcyclohexa-1,3-diene-1-carbaldehyde) is the primary constituent of saffron and has the structure

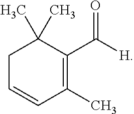

In an aspect, pure safranal, that is safranal which has been separated from the non-safranal components of saffron, is used in the compositions and methods described herein.

Hydroxytyrosol (4-(2-hydroxyethyl)benzene-1,2-diol) is a phenylethanoid found in olive oils and wine having the structure

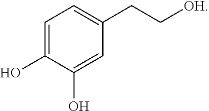 [SMILES: OCCc1ccc(O)c(O)c1]

In an aspect, pure hydroxytyrosol, that is hydroxytyrosol which has been separated from the non-hydroxytyrosol components of a food source such as olive oil or olives, is used in the compositions and methods described herein.

Menaquinone-4 also known as menatetrenone (3-methyl-2-[(2Z,6E,10E)-3,7,11,15-tetramethylhexadeca-2,6,10,14-tetraenyl]naphthalene-1,4-dione) is a vitamin K2 homolog having the structure

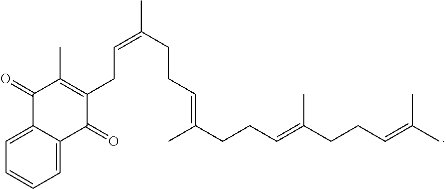 [SMILES: CC(C)=CCC/C(C)=C/CC/C(C)=C/CC/C(C)=C\CC1=C(C)C(=O)c2ccccc2C1=O]

In an aspect pure menaquinone-4 can be synthesized and used in the compositions and methods described herein.

Menaquinone-7 also known as 2-[(2E,6E,10E,14E,18E,22E)-3,7,11,15,19,23,27-heptamethyloctacosa-2,6,10,14,18,22,26-heptaenyl]-3-methylnaphthalene-1,4-dione. Menaquinone-7 is a vitamin K2 like menaquinone-4. Menquinone-7 has 7 isoprene units compared to menaquinone-4 which has 4 isoprene units. Menquinone-7 has a longer half-life in the human body than menaquinoe-4. In an aspect, menaquinone-7 can be produced by fermentation of Bacillus subtilis (natto) and purified and used in the compositions and methods described herein.

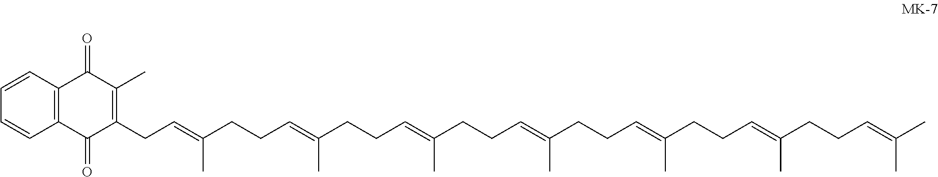 [SMILES: CC(C)=CCC/C(C)=C/CC/C(C)=C/CC/C(C)=C/CC/C(C)=C/CC/C(C)=C/CC/C(C)=C/CC1=C(C)C(=O)c2ccccc2C1=O]

Oleocanthal, also known as 2-(-4-hydroxyphenyl)ethyl (3S,4E)-4-formyl-3-(2-oxoethyl)hex-4-enoate), is a phenylethanoid found in extra virgin olive oil. The structure of oleocanthol is shown below.

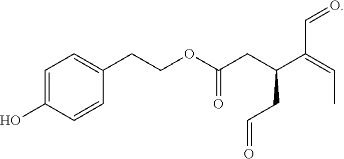 [SMILES: C/C=C(/C=O)[C@@H](CC=O)CC(=O)OCCc1ccc(O)cc1]

In an aspect, pure oleocanthol, that is pure oleocanthol which has been separated from the non-oleocanthol components of a food source such as olive oil, is used in the compositions and methods described herein. Pure oleocanthol is an oil.

Peptide Ins-1 B9-23 19CAM22R-E SHLVEALYLVCGEEG (SEQ ID NO: 1) is modified by treating the peptide with iodoacetamide to carboxyamidomethylate the cysteine residue.

Peptide Ins-1 B12-23-19CAM is VEALYLVCGERG (SEQ ID NO: 2) is modified by treating the peptide with iodoacetamide to carboxyamidomethylate the cysteine residue.

Peptide sequence Ins-1 B12-23-19CAM22R-E having sequence VEALYLVCEGEEG (SEQ ID NO: 3) is modified by treating the peptide with iodoacetamide to carboxyamidomethylate the cysteine residue.

As used herein, a “pure” compound is a compound that is 98%, 99% or greater than 99% pure.

Administration of the compound is preferably started as soon as practical after birth, preferably between birth and 6 months of age. In an aspect, administration of the compound is started between four and six months of age. In another aspect, administration is continued until the child is 1, 2, 3, 4, 5, 6, 7, or 8 years old.

In an aspect, administration is chronic administration. As used herein chronic administration is administration over a long duration of time such as a month, several months, or even years. Administration can be daily, every other day, weekly, monthly, or less frequent. In an aspect, the frequency of the administration is daily to monthly, for a period of at least age 2 to 8 years.

The dose of the compound depends upon the particular compound to be administered. All of the compounds are generally regarded as safe for humans, so the dose would be within the dose that is known in the art to be safe for humans, particularly human children. Exemplary dosages can be readily determined by one of ordinary skill in the art based on dosing in the art. In an aspect, the dosages of the compounds described herein, i.e., the daily doses of treatments that significantly lowered the rates of diabetes were weights or micromoles of chemicals that were lower than weights or micromols of antibiotics or other medicines given to human children up to about 14 years of age.

In an aspect, the human child is at risk of developing type 1 diabetes. In an aspect, the human child is at increased risk of developing type 1 diabetes because a parent or sibling has type 1 diabetes and/or the child possesses high risk type 1 diabetes HLA alleles such as either HILA-DRB1*03 (DR3) or HLA-DRB1*04 (DR4) or both alleles.

In an aspect, administering reduces the risk of type 1 diabetes in the child. Preferably, the child reaches age 5, age 8-10 or even age 25 without developing type I diabetes. Measurable tests for type I diabetes include antibodies to insulin, for example, which can be detected months or even years before an increase in blood sugar is detected. Other tests for type 1 diabetes include blood sugar levels and hemoglobin A1c levels.

In an aspect the agent or peptide is administered by subcutaneous injection to the human child starting at birth and continuing until the child is 6 months to 8 years of age.

In an aspect, the compound is administered in an infant formula, as a supplement to add to an infant formula, or a multivitamin. Preferably, infant formula is a substitute for human milk which meets the full nutritional needs of an infant under about 12 months of age. Infant formulas are well known in the art and include protein, fats, carbohydrates, vitamins, minerals and other nutritional additives such as prebiotics and probiotics. Infant formulas can be in ready to use liquid form or powders that must be reconstituted prior to use, for example. Supplements to add to an infant formula can be in liquid or powder form. For older children, the compound can be administered as a multivitamin in liquid or solid form, such as a swallowable, chewable or gummy vitamin. Multivitamins for children can include, for example, Vitamins A, D, E, C, B1, B2, B3, B5, B6, B12, folic acid, iron, magnesium and zinc.

In another aspect, a method of treating a pregnant human or a nursing human mother who has recently given birth to an infant comprises administering to the pregnant human and/or to the nursing human mother a compound selected from epigallocatechin gallate (EGCG), epicatechin (EC), epigallocatechin (RGC), epicatechin gallate (ECG), sulforaphane, olive oil, phylloquinone, quercetin, safranal, hydroxtyrosol, menaquinone-4, trans menaquinone-7, oleocanthal, peptide Ins-1 B9-23 19CAM22R-E, peptide Ins-1 B12-23-19CAM, peptide Ins-1 B12-23-19CAM22R-Esafranal, or a combination of any of the foregoing, wherein administration to the pregnant mother is begun during the final two months of gestation.

In an aspect, the pregnant human or nursing human mother, the father, or a sibling has type 1 diabetes. Having a parent or sibling with type 1 diabetes is associated with an increased risk for developing type 1 diabetes.

In an aspect, the compound is administered in a vitamin or a dietary supplement. As used herein, a multivitamin is a supplement containing multiple vitamins and/or minerals typically to ensure consumption of a recommended daily allowance of nutrients. Multivitamins can be in liquid or solid form, such as a chewable or gummy vitamin. A dietary supplement is intended to add or supplement the diet and is not a conventional food. Dietary supplements can be in liquid or solid form, such as a swallowable, chewable or gummy vitamin, a drink, a bar, a powder, a gel and the like.

In an aspect, the multivitamin is a prenatal vitamin. Prenatal vitamins are similar to regular multivitamins except that they include folic acid to help prevent neural tube defects as well as higher levels of other vitamins and minerals than typical multivitamins.

In another aspect, an insulin B chain mimotope amino acid peptide is Ins-1 B9-23 19CAM22R-E having the sequence SHLVEALYLVCGEEG (SEQ ID NO: 1) modified by treating the peptide with iodoacetamide to carboxyamidomethylate the cysteine residue, peptide Ins-1 B12-23-19CAM having the sequence VEALYLVCGERG (SEQ ID NO: 2) modified at modified by treating the peptide with iodoacetamide to carboxyamidomethylate the cysteine residue, or peptide sequence Ins-1 B12-23 19CAM22R-E having sequence VEALYLVCEGEEG (SEQ ID No.3) modified by treating the peptide with iodoacetamide to carboxyamidomethylate the cysteine residue.

Also included is a pharmaceutical composition comprising the foregoing peptides and a pharmaceutically acceptable carrier or excipient.

In an aspect, the peptides can be used to vaccinate a child via subcutaneous injection. In an aspect, the administering is done as an addition to an ordinary course of infant injections.

As used herein, “pharmaceutical composition” means therapeutically effective amounts of the compound together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anesthetic preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The invention is further illustrated by the following non-limiting examples.

### EXAMPLES

### Materials and Methods

Mouse care: NOD/ShiLtJ mice were from The Jackson Laboratory (Bar Harbor, ME, USA). Mice were housed under standard conditions (20-24° C., humidity 30-70%, lighting regimen 12 h light/12 h dark) in the University of Wisconsin Biomedical Research Model Service that is a specific pathogen free level 1 facility. Mouse pups were caged with the breeder parents until age 21 days and were fed the 2919 Irradiated Teklad Global Diets® Protein-Free Extruded Rodent diet. After weaning, the mouse pups were housed separately from their parents and fed the 2920x Irradiated Teklad Global Diets® Protein-Free Extruded Rodent diet. Mice had access to acidified water or water containing a chemical and/or green tea ad libitum. All procedures were approved by the Research Animal Resources and Compliance veterinary care authority of the University of Wisconsin, Madison.

Mouse treatments: Mice were given chemicals by intraperitoneal injection and/or by gavage and/or in drinking water. Water-soluble chemicals were administered in a solution of sodium chloride adjusted to have a final osmolality of 0.5-0.9%. Lipid soluble chemicals were dissolved in olive oil, peanut oil, corn oil, Mazola® oil or canola oil. Olive oil and peanut oil were from Acros Organics (catalog numbers 41654 and 41685, respectively). Modified insulin peptides were given by the intraperitoneal route in 5% dimethyl sulfoxide:/95% 0.9% saline.

Starting at age 2 to 5 days mice were given injections on every third or fourth day for 2 to 4 weeks (usually 2 to 2.5 weeks) for the first 4 or 6 injections, and thereafter twice per week on Mondays and Thursdays until up to age 100 days. In some instances, pregnant and nursing mice were given chemicals in drinking water starting at about the middle or late pregnancy continuing during nursing and also continuing for various lengths of time to the offspring with or without additional intraperitoneal injections of chemicals. In other instances, chemicals were delivered by intraperitoneal injection into pregnant mice and continued after delivery into the mouse pups. Chemicals, for example EGCG, were also delivered in drinking water starting before birth to the pregnant mouse mother and/or continuing and/or starting ad libitum to the mice after birth usually until at least 100 days of age or throughout life.

Chemicals given in distilled drinking water were green tea (as a source of EGCG and other catechins) (Lipton® 100% Natural Green Tea). The tea was prepared with 6 bags of tea per 500 ml water (The green tea was from the Lipton company because this green tea is reported to have higher concentrations of catechins than green tea sold by other companies). The tea water was given alone and also in combination with acetaminophen 2 g/l in the same water.

Assessment of diabetes: Diabetes was judged to be permanent when tail blood glucose values persistently exceeded 250 mg glucose/dl (13.9 mM). Diabetic mice were euthanized when blood glucose consistently exceeded 375 mg glucose/dl (20.8 mM). The Jackson Laboratory website shows that the average blood sugar values of two different samples of nondiabetic (prior to diabetes onset) NOD/ShiLtJ female mice equaled 160+/−17 and 191+/−14 mg glucose/dl (mean+/−SEM). In the current study, the final average blood glucose values of the diabetic mice associated with the various treatments were very high with a low of 603 and a high of 711 mg glucose/dl and the actual average blood glucose values of the nondiabetic mouse values were a low of 143 and a high of 182 mg glucose/dl blood as shown in Tables 1, 2 and 3. In most cases nondiabetic mice were euthanized at 40-41 weeks of age. Statistical significances of the frequencies of diabetes in the mice receiving test treatments were compared to the rates of diabetes in mice receiving control treatments and were calculated with the Chi square test and with the Chi square test with Yate's correction.

Administration of treatments to NOD T1D-prone mice: Similar to other studies of type 1 diabetes in NOD mice, only treatments of female mice were studied because female NOD mice develop a higher rate of diabetes than male NOD mice (about 80% in female mice and 50% in male mice at 30 weeks of age according to Jackson Laboratory data). In most of the studies, control solutions and the other treatments were administered to the mice until they were 100 days old. Mice without diabetes were euthanized at 40-41 weeks of age. Diabetic mice were euthanized when their blood glucose levels reached a level that met the criteria for diabetes.

Administration of water-soluble chemicals serving as controls to NOD mice: Control solutions were the vehicles in which chemicals were dissolved. Control solutions were usually administered to mouse litters from the same shipments as the litters receiving test chemicals and/or litters from the same parents as the litters receiving test chemicals and were administered during the same month and year as treatments to the litters receiving test treatments. Statistical probabilities between the rates of diabetes in the litters receiving test treatments were compared to litters simultaneously receiving control treatments.

Intraperitoneal injections of water-soluble chemicals were given in normal saline (0.9% sodium chloride). Intraperitoneal injection of normal saline alone at 41 weeks of age resulted in a diabetes rate of 81%. Other test chemicals that did not significantly lower the rate of diabetes compared to saline injections served as additional controls for comparison with the treatments that did lower the rate of diabetes. Because of its toxicity to the mice CoQ0 was given at a very low level. Mice given 20 mol CoQ0/kg until ages 58 to 61 days had an 83% frequency of diabetes at 34 weeks of age. Mice receiving tea with acetaminophen in water orally had an incidence of diabetes of 88% at 41 weeks of age. Mice given intraperitoneal tiopronin and caffeic acid had an incidence of diabetes of 74% at 40 weeks of age and 65% at 34 weeks of age respectively (Table 1)

If a chemical that was associated with a rate of diabetes of 50% or higher, even if the rate was statistically different from the rate of diabetes in the litters receiving a control treatment, the treatment was not further pursued as a diabetes preventive treatment even though it may have been used as a negative control.

### Example 1: Treatments with Water Soluble Chemicals that Lowered the Rate of Diabetes

EGCG: Table 1 shows that in mice given EGCG alone in saline the rate of diabetes was lowered to 35% compared to 81% in mice that received saline. Similar high rates of diabetes were observed in mice receiving other control treatments. When hydroxytyrosol was combined with the EGCG injection or oral tea water was given to the mice that were receiving EGCG, the rates of diabetes in these two groups were not significantly different from the rate of diabetes in the mice that received EGCG alone. This indicates that the diabetes preventative action was due to EGCG.

EGCG dissolved in saline was given intraperitoneally twice per week for two weeks starting at age 3 to 5 days until ages 23-28 days, and then once per week thereafter until age 59-63 days or 93-95 days. At age 41 to 44 weeks, only 35% of the EGCG-treated mice had diabetes. (The rates of diabetes with treatments ending after 59-63 days and 93-95 days of EGCG treatment were similar at 38% and 31%, respectively.) Another separate group of mice were given EGCG via the intraperitoneal route that was stopped at age 59-60 days. This group had a diabetes rate of 33% at 40 weeks of age (Table 1).

Mouse pups were also given EGCG intraperitoneally starting twice per week starting at age 3 or 4 days until 17 or 18 days of age, and then once per week thereafter until age 56-60 or 98 days plus green tea instead of water. In this group the mouse litters received tea water starting 7-10 days before delivery to the pregnant mother and continuing after birth to the breeding pairs with nursing pups in the same cage and then to the pups continuing throughout the lives of the animals. At age 41 weeks, only 30% of the NOD mice had diabetes. (The rates of diabetes in the mice receiving EGCG for 59-60 days and 98 days were similar at 29% and 30%, respectively.) Tea water alone gave a diabetes rate (77%) similar to the rate of the saline control (Table 1).

A third group of mice were given EGCG along with hydroxytyrosol intraperitoneally starting at 2 to 4 days of age with the treatment stopped at 97 or 98 days of age. (Hydroxytyrosol was given because it is a component of olive oil that lowered the rate of diabetes. See also Table 3 for effects of olive oil.) At 41 weeks of age the rate of diabetes in this group was 41% (Table 1). Although the diabetes rate with hydroxytyrosol alone control (59%) was significantly lower (P=0.02 (Chi square test) and P=0.04 (Yate's chi square test)) than with the saline control (81%), the hydroxytyrosol did not increase the diabetes lowering effect of EGCG.

The above results with three different groups of mice that received EGCG starting right after birth indicate that the diabetes preventative effect was due to EGCG.

EGCG was also administered to the mice by the intraperitoneal route starting at 21 days of age which is after the mice were weaned from their mother's milk and until the mice were 99 days of age. This treatment lowered the rate of diabetes to 29% (Table 1) and indicates that treatment with EGCG can prevent diabetes when started after weaning from breast milk which for most human infants occurs at or before six months of age.

A low concentration (1 mM) of EGCG was also administered to the mice in drinking water starting at 1 to 4 days after birth. This lowered the rate of diabetes to 40% (Table 1). A high concentration (20 mM) of EGCG in drinking water starting at 1 to 3 days of age has kept the rate of diabetes in the mice at zero at 20 to 33 weeks of age (Table 1). These mice are being kept alive until age 40 weeks or until they develop severe diabetes.

Mice given EGCG (10 mM) in drinking water along with olive oil via the intraperitoneal route starting at age 2 to 4 days and stopping at age 96-99 days of age. At 40 weeks of age the rate of diabetes was 41% (Table 1).

Mice treated with EGCG via the intraperitoneal route until they were weaned at age 21 days of age and then subsequently by gavage until age 97-98 days of age had a diabetes rate of 50% (Table 1).

Sulforaphane: Sulforaphane was given to the mouse pups by the intraperitoneal route during nursing and then by gavage starting after weaning at 21 days of age and continued until 55 to 62 days of age. Sulforaphane gave a frequency of diabetes of 42% at 35 weeks of age (Table 1), which is significantly lower than the saline control.

### Example 2: Administration of Insulin Peptides with Modified Cysteine Residues

Peptides with a modified cysteine residue decrease the incidence of diabetes. Modified insulin peptides were dissolved in 5% dimethyl sulfoxide/95% saline. An insulin peptide based on a mimotope of the native insulin B chain amino acid sequence 12 to 23 mimotope (where glutamate is substituted for the native arginine residue at position 22) was previously designed and tested. This peptide mimotope sequence (VEALYLVCGEEG; SEQ ID NO: 4) prevented type 1 diabetes in the NOD mouse. However, the same peptide has been reported to precipitate rather than prevent type 1 diabetes in the NOD mouse. A similar insulin B chain 9-23 sequence mimotope peptide (SHLVEALYLVCGEEG; SEQ ID NO: 1) was modified by treating the peptide with iodoacetamide to carboxyamidomethylate the cysteine residue at position 19. This modified peptide is denoted as Ins-1 B9-23 19CAM22R-E. Ins-1 B9-23 19CAM22R- was administered once per week to mice in 20 μg doses for the first 3 weeks of life and then 40 ug doses once per week until the mice were 100 to 110 days old. At 40 weeks of age only 17% of the mice had developed diabetes (Table 2).

NOD mice were also given a peptide consisting of the insulin B chain native amino acid sequence 12 to 23 (VEALYLVCGERG; SEQ ID NO: 2) with the cysteine at position 19 carboxyamidomethylated by reacting it with iodoacetamide. This peptide is denoted as Ins-1 B12-23-19CAM. NOD mice were further given peptide sequence Ins-1 B12-23 19CAM22R-E having sequence VEALYLVCEGEEG (SEQ ID NO: 3) modified by reacting the peptide with iodoacetamide to carboxyamidomethylate their cysteine residue. This peptide is denoted as Ins-1 B12-23 19CAM22R-E. The rate of diabetes in mice given these peptides was lowered to 47% (Table 2).

### Example 3: Administration of Olive Oil and Chemicals Dissolved in Olive Oil that Lowered the Incidence of Type 1 Diabetes

Olive oil: Olive oil or peanut oil were used as a vehicle for water-insoluble chemicals. Interestingly, olive oil alone administered by intraperitoneal injection starting at ages 3 to 5 days until the mice were 99 days of age lowered the rate of diabetes to 37% (p=0.0001 and p=0.0002 vs. saline alone (Chi square and Yate's chi square, tests respectively) at 41 weeks of age (Table 3). Certain chemicals that when dissolved in olive oil and given by the intraperitoneal route were associated with even lower rates of diabetes at 40 weeks of age than olive oil alone even though these lower rates of diabetes were not statistically different from the rate of diabetes with olive oil alone. These were phylloquinone (2,000 to 3,5000 μmol/kg/IP injection) in olive oil (diabetes rate 21%), quercetin in olive oil (diabetes rate 26%), safranal in olive oil (diabetes rate 32%), menaquinone-4 plus quercetin in olive oil (diabetes rate 19%) and menaquinone-4 in olive oil (diabetes rate 0%) at 40 weeks of age (Table 3). Olive oil given to mouse pups by the intraperitoneal route while they were nursing, and then after 20 days of age by gavage, gave a rate of diabetes of 41% at 40 weeks of age (Table 3). Safranal in olive oil was also given to the mouse pups by intraperitoneal injection until they weaned from mother's milk at age 21 days followed by delivery by gavage until age 98 days. This resulted in a rate of diabetes of 36% at 40 weeks of age (Table 3).

Menaquinone-4 was given to the mice for 30 days and also 60 days of age by intraperitoneal injection. This resulted in rates of diabetes of 32% and 4%, respectively, at 20-24 weeks and 34-36 weeks of age. Menaquinone-4 was also given by intraperitoneal injection until weaning at 21 days of age followed by delivery by gavage until age 98 days. This resulted in a rate of diabetes of 25% at age 24-30 weeks. (Table 3). These studies are being continued until nondiabetic mice are 40 weeks old.

Olive oil administered by the intraperitoneal route starting after weaning at age 20 days of age and until the mice were 97 to 99 days of age lowered the rate of diabetes to 54%. This rate was statistically different (0.02, Chi square test; 0.04, Yate's chi square test) from the rate with the saline control intraperitoneal injections, but not as low as the rate with olive oil given by the intraperitoneal route starting at age 3 to 5 days (37%) (Table 3). This indicates that olive oil's diabetes preventative action begins early in life soon after birth. This suggests that if olive oil were to be administered to humans to prevent type 1 diabetes it should be started as early in life as is practical. Oleocanthal is exclusively found in extra-virgin olive oil. When pure oleocanthal was administered to the diabetes-prone mice the rate of diabetes was lowered to 35% (Table 3) indicating that oleocanthal could be a major factor responsible for diabetes prevention by olive oil.

Olive oil contains numerous antioxidants. Hydroxytyrosol is a major polyphenol and antioxidant in olive oil, and it was administered to the mice in saline. As shown in Table 1, it gave a diabetes rate of 59% that was significantly lower than the diabetes rate with the saline control (81%, p=0.02 and 0.04, Chi square and Yate's chi square, respectively (Table 1)) indicating that hydroxytyrosol may account for a small part of the diabetes-lowering action of olive oil.

Peanut oil: Several water-insoluble chemicals were dissolved in peanut oil administered into the peritoneal cavity of the mice. When the animals receiving only peanut oil as a control were euthanized at ages 34 to 37 weeks, the final rate of diabetes was 67% (not significant vs. the saline alone control) (Table 3). When five different chemicals were individually dissolved in peanut oil and administered to the mice, the rate of diabetes averaged 56+/−8.3% (mean+/−SE, range of diabetes 47-71%, N=5 different chemicals) (Not all shown in Table 3.) Animals without diabetes were euthanized at 38 to 41 weeks of age. The diabetes rates were not significant vs. diabetes rate with peanut oil alone. Because peanuts are associated with severe allergies in humans, chemicals administered in peanut oil as a carrier were not pursued further.

These experiments will be repeated with corn oil, Mazola® oil and canola oil used to dissolve the various compounds.

TABLE 1
WATER SOLUBLE CHEMICALS (EGCG BY ITSELF OR COMBINED WITH OTHER AGENTS OR SULFORAPHANE
BY ITSELF) LOWER THE INCIDENCE OF TYPE 1 DIABETES IN THE NOD MOUSE

| | | | | | | Final Blood Glucose | | p Value % Diabetes |
|---|---|---|---|---|---|---|---|---|
| | Dosage of | Route of | Carrier | | | (mean ± SE) | | with Chemical vs |
| | chemical | Chemical | for | | Diabetes | non- | | Carrier (Chi square, |
| Chemical(s) | (μmol/kg) | delivery | delivery | N | (%) | diabetic | diabetic | Yates chi square) |
| Saline control | | IP | | 37 | 81 | 156 ± 12 | 629 ± 19 | |
| EGCG | 50 | IP | saline | 26 | 35 | 159 ± 5 | 685 ± 34 | 0.0006, 0.0015 |
| | | | | | | | | vs saline, 0.005, |
| | | | | | | | | 0.01 vs CoQ0 |
| EGCG + Tea water | 50 | EGCG: IP, | EGCG: | 37 | 30 | 143 ± 4 | 654 ± 36 | 0.00003, 0.0001 |
| | | Tea: oral | saline | | | | | vs saline, 0.002, |
| | | | | | | | | 0.006 vs CoQ0 |
| EGCG | 50 | IP | ¾ | 28 | 29 | 166 ± 4 | 605 ± 59 | 0.000007, 0.00002 |
| (started post wean) | | | saline | | | | | vs saline |
| EGCG | 250 | gavage | water | 24 | 25 | | | 24-27 weeks |
| (started post wean) | | | | | | | | |
| EGCG | 1 mM | oral | drinking | 20 | 40 | 172 ± 4 | 708 ± 24 | 0.0009, 0.002 vs |
| | | | water | | | | | saline |
| EGCG | 20 mM | oral | drinking | 14 | 0 | | | 20-33 weeks |
| | | | water | | | | | |
| EGCG | EGCG: 10 mM | EGCG: oral | EGCG: | 22 | 41 | 184 ± 24 | 656 ± 24 | 0.002, 0.004 vs |
| and Olive Oil | Olive Oil: | Olive Oil: | drinking | | | | | saline |
| | 40 μl/mouse | IP | water | | | | | |
| | (20-1.6 ml/kg) | | | | | | | |
| EGCG | 50 | IP | saline | 30 | 33 | 145 ± 4 | 663 ± 57 | 0.00002, 0.00005 |
| (60 day treatment) | | | | | | | | vs saline |
| EGCG + | EGCG: 50 | IP | ¾ | 27 | 41 | 168 ± 9 | 674 ± 22 | 0.01, 0.02 vs saline |
| Hydroxytyrosol | Hydroxytyrosol: | | saline | | | | | |
| Hydroxytyrosol | 650 325 first | IP | ¾ | 37 | 59 | 173 ± 12 | 672 ± 24 | 0.02, 0.04 vs saline |
| | 5 weeks, 650 | | | | | | | |
| control | thereafter | | saline | | | | | |
| Tea water control | | oral | | 22 | 77 | 183 ± 6 | 624 ± 22 | NS vs saline |
| EGCG | IP: 50 | IP til wean, | saline | 24 | 50 | 200 ± 12 | 624 ± 33 | 0.01, 0.02 vs saline |
| | Gavage: 100 | then Gvg | | | | | | |
| Sulforaphane | 200 | IP til wean, | ¾ | 19 | 42 | 154 ± 7 | 619 ± 37 | 0.001, 0.004 vs |
| | | then Gvg | saline | | | | | saline |
| CoQ0 control | 20 | IP | saline | 12 | 83 | 183 ± 28 | 649 ± 53 | NS vs saline |
| Tea + | Acetaminophen | oral | | 17 | 88 | 154 ± 15 | 692 ± 22 | NS vs saline |
| Acetaminophen | in water = 13 | | | | | | | |
| water | mM (2 mg/ml) | | | | | | | |
| Tiopronin | 200 | IP | saline | 23 | 74 | 175 ± 12 | 685 ± 19 | NS vs saline |
| Caffeic Acid | 100 | IP | saline | 20 | 65 | 178 ± 15 | 611 ± 32 | NS vs saline |

TABLE 2
PEPTIDES THAT LOWERED THE INCIDENCE OF TYPE 1 DIABETES IN THE NOD MOUSE

| | | | | | | Final Blood Glucose | | p Value Peptide |
|---|---|---|---|---|---|---|---|---|
| | | Route of | | | | (mean ± SE) | | vs Carrier (Chi |
| | Dosage of | Chemical | Carrier for | | Diabetes | non- | | square, Yates |
| Chemical(s) | chemical | delivery | delivery | N | (%) | diabetic | diabetic | chi square) |
| Saline control | | IP | | 37 | 81 | 156 ± 12 | 629 ± 19 | |
| Ins B9-23 | 20 ug, then | IP | 5% DMSO/ | 12 | 17 | 159 ± 6 | 681 ± 82 | 0.00005, 0.0002 |
| 19CAM22R-E | 40 μg after 3 | | 95% saline | | | | | vs saline |
| | weeks | | | | | | | |
| Ins B12-23 | 20 ug, then | IP | 5% DMSO/ | 19 | 47 | 160 ± 6 | 679 ± 32 | 0.0002, 0.02 |
| 19CAM | 40 μg after 3 | | 95% saline | | | | | vs saline |
| | weeks | | | | | | | |
| Ins B12-23 | 40 μg twice/ | IP | 5% DMSO/ | 24 | 54 | 166 ± 7 | 569 ± 70 | 0.02, 0.049 vs |
| 19CAM22R-E | week for two | | 95% saline | | | | | saline 0.018, |
| | weeks then | | | | | | | 0.04 vs 5% |
| | once per | | | | | | | DMSO |
| | week until | | | | | | | |
| | 14 weeks old | | | | | | | |

TABLE 3
OLIVE OIL ALONE OR CHEMICALS IN OLIVE OIL THAT LOWERED
THE FREQUENCY OF TYPE 1 DIABETES IN THE NOD MOUSE

| | | | | | | Final Blood Glucose | | P Value Treatment |
|---|---|---|---|---|---|---|---|---|
| | Dosage of | Route of | | | | (mean ± SE) | | vs Carrier (Chi |
| | chemical | Chemical | Carrier for | | Diabetes | non- | | square, Yates |
| Chemical(s) | (μmol/kg) | delivery | delivery | N | (%) | diabetic | diabetic | chi square) |
| Saline control | | IP | | 37 | 81 | 156 ± 12 | 629 ± 19 | |
| Peanut Oil control | Control | IP | | 24 | 67 | 177 ± 12 | 711 ± 19 | NS vs saline |
| Olive oil control | 40 μl/mouse | IP | | 41 | 37 | 165 ± 5 | 675 ± 16 | 0.00007, 0.0002 |
| | (20-1.6 ml/kg) | | | | | | | vs saline 1.2 × 10⁻⁷, |
| Phylloquinone | 2000-3500 | IP | olive oil | 42 | 21 | 163 ± 5 | 647 ± 27 | 4 × 10⁻⁷ vs saline |
| | | | | | | | | NS vs olive oil |
| Phylloquinone | 110 | IP | olive oil | 29 | 38 | 174 ± 5 | 648 ± 37 | 0.001, 0.002 vs |
| | | | | | | | | saline |
| | | | | | | | | NS vs olive oil |
| Phylloquinone | 2000-3500 | IP | peanut oil | 20 | 40 | 167 ± 10 | 646 ± 40 | 0.002, 0.004 vs |
| | | | | | | | | saline |
| | | | | | | | | NS vs peanut oil |
| Quercetin | 200 | IP | olive oil | 23 | 26 | 178 ± 7 | 610 ± 56 | 0.0004, 0.0001 |
| | | | | | | | | vs saline |
| | | | | | | | | NS vs olive oil |
| Quercetin | 200 | IP | peanut oil | 17 | 47 | 171 ± 11 | 651 ± 25 | 0.01, 0.04 vs saline |
| | | | | | | | | NS vs peanut oil |
| Menaquinone-4 | 200 | IP | peanut oil | 18 | 50 | 164 ± 9 | 706 ± 25 | 0.02. 0.04 |
| | | | | | | | | vs saline |
| | | | | | | | | NS vs peanut oil |
| Menaquinone-4 | 200 | IP | olive oil | 20 | 0 | 172 ± 15 | | 0, 0.00000003 vs |
| | | | | | | | | saline 0.002, 0.005 |
| | | | | | | | | vs olive oil |
| Menaquinone-4 | 200 | IP | olive oil | 24 | 4 | | | 34-36 weeks |
| (60 day treatment) | | | | | | | | |
| Menaquinone-4 | 200 | IP | olive oil | 25 | 32 | | | 20-40 weeks |
| (30 day treatment) | | | | | | | | |
| Menaquinone-4 | 200 | IP til | olive oil | 32 | 25 | | | 24-30 weeks |
| | | wean, | | | | | | |
| | | then gvg | | | | | | |
| Menaquinone-4 + | 200 + 200 | IP | olive oil | 16 | 19 | 161 ± 4 | 505 ± 100 | 0.00003, 0.0001 |
| Quercetin | | | | | | | | vs saline |
| | | | | | | | | NS vs olive oil |
| Menaquinone-4 + | 200 + 200 | IP | peanut oil | 17 | 71 | 164 ± 16 | 603 ± 43 | NS vs saline |
| Quercetin | | | | | | | | NS vs peanut oil |
| Olive Oil | 3.6-10.5 ml/kg | Oral | | 29 | 41 | 157 ± 4 | 644 ± 40 | 0.002, 0.004 vs |
| (6.3 ml/kg IP to | to pups post | Gavage | | | | | | saline |
| nursing mothers. | wean | | | | | | | |
| Oral to pups post | | | | | | | | |
| wean.) | | | | | | | | |
| Safranal | 2000 | IP | olive oil | 19 | 32 | 159 ± 5 | 606 ± 60 | 0.0002, 0.0005 |
| | | | | | | | | vs saline |
| | | | | | | | | NS vs olive oil |
| Safranal | 1500 | IP til | olive oil | 25 | 36 | 172 ± 8 | 595 ± 36 | 0.0003, 0008 vs |
| | | wean, | | | | | | saline |
| | | then gvg | | | | | | NS vs olive oil |
| Phylloquinone | 3100-4000 | IP | olive oil | 28 | 36 | 156 ± 6 | 677 ± 20 | 0.0002, 0.0005 |
| (started post wean) | | | | | | | | vs saline |
| | | | | | | | | NS vs olive oil |
| Olive Oil | | IP | olive oil | 24 | 54 | 175 ± 8 | 682 ± 22 | 0.02, 0.04 vs saline |
| (started post wean) | | | | | | | | NS vs olive oil |
| Oleocanthal (oil) | 50 | IP til | None | 20 | 35 | 178 ± 7 | 560 ± 61 | 0.0005, 0.004 vs |
| | | wean, | | | | | | saline |
| | | then Gvg | | | | | | |

** All non-diabetic mice are kept alive until age 40 weeks. In Tables 1 and 3 mice without diabetes and with their current ages stated along with no statistical data described in the column at the far right will be kept alive until age 40 weeks.

### Definitions and Abbreviations

IP, intraperitoneal.
CoQ0, 2,3-dimethoxy-5-methyl-1,4-benzoquinone.
EGCG, epigallocatechin gallate.
DMSO, dimethyl sulfoxide.
NS, not significant.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating a human child, comprising administering to the human child a compound selected from epigallocatechin gallate (EGCG), epicatechin (EC), epigallocatechin (RGC), epicatechin gallate (ECG), sulforaphane, olive oil, phylloquinone, quercetin, safranal, hydroxtyrosol, menaquinone-4, menaquinone-7, oleocanthal, peptide Ins-1 B9-23 19CAM22R-E, peptide Ins-1 B12-23-19CAM, peptide Ins-1 B12-23 19CAM22R-E, or a combination of any of the foregoing, wherein administration of the treatment is started between birth and six months of age.

2. The method of claim 1, wherein the frequency of the administration is daily to monthly.

3. The method of claim 1, wherein treatment continues through a period of at least 2 years up to 8 years of age.

4. The method of claim 1, wherein the human infant is at increased risk of developing type 1 diabetes because a parent or sibling has type 1 diabetes and/or the infant possesses high risk type 1 diabetes HLA alleles such as either HLA-DRB1*03 (DR3) or HLA-DRB1*04 (DR4) or both alleles.

5. The method of claim 1, wherein the administering reduces the risk of type 1 diabetes in the child.

6. The method of claim 1, wherein the compound is administered in an infant formula, a supplement to be added to an infant formula, a multivitamin, or by subcutaneous injection.

7. A method of treating a pregnant human or a nursing human mother who has recently given birth to an infant, comprising administering to the pregnant human and/or to the nursing human mother a compound selected from epigallocatechin gallate (EGCG), epicatechin (EC), epigallocatechin (RGC), epicatechin gallate (ECG), sulforaphane, olive oil, phylloquinone, quercetin, safranal, hydroxtyrosol, menaquinone-4, trans menaquinone-7, oleocanthal, peptide Ins-1 B9-23 19CAM22R-E, peptide Ins-1 B12-23-19CAM, peptide Ins-1 B12-23-19CAM22R-Esafranalor a combination of any of the foregoing, wherein administration to the pregnant mother is begun during the final two months of gestation.

8. The method of claim 7, wherein the frequency of the administration is daily to monthly,

9. The method of claim 7, wherein treatment continues through a period of at least 2 years up to 8 years of age.

10. The method of claim 7, wherein the pregnant human or a nursing human mother's mother, father, or a sibling has-type 1 diabetes.

11. The method of claim 7, wherein the compound is administered in a multivitamin or a dietary supplement.

12. The method of claim 7, wherein the administering the compound reduces the risk of type 1 diabetes in the child.

13. The method of wherein the compound is administered in a prenatal vitamin.

14. An insulin B chain mimotope peptide Ins-1 B9-23 19CAM22R-E having sequence SHLVEALYLVCGEEG (SEQ ID NO: 1) modified by treating the peptide with iodoacetamide to carboxyamidomethylate the cysteine residue, peptide Ins-1 B12-23-19CAM having sequence VEALYLVCGERG (SEQ ID NO: 2) modified at modified by treating the peptide with iodoacetamide to carboxyamidomethylate the cysteine residue, or peptide Ins-1 B12-23 19CAM 22R-E having sequence VEALYLVCEGEEG (SEQ ID No.3) modified by treating the peptide with iodoacetamide to carboxyamidomethylate the cysteine residue.

15. A pharmaceutical composition comprising the modified insulin peptide of claim 14 and a pharmaceutically acceptable excipient.

16. A method of vaccinating a child comprising administering to the child the pharmaceutical composition of claim 15.

17. The method of claim 16, wherein vaccinating is done as a series of injections.

18. The method of claim 16, wherein the administering is done as an addition to an ordinary course of infant injections.


SEQUENCE LISTING (4 sequences)

SEQ ID NO: 1 - protein, 15 aa, synthetic construct
  MOD_RES 11: note = Carboxyamidomethylated Cysteine
SHLVEALYLVCGEEG

SEQ ID NO: 2 - protein, 12 aa, synthetic construct
  MOD_RES 8: note = Carboxyamidomethylate Cysteine
VEALYLVCGERG

SEQ ID NO: 3 - protein, 13 aa, synthetic construct
  MOD_RES 8: note = Carboxyamidomethylate Cysteine
VEALYLVCEGEEG

SEQ ID NO: 4 - protein, 12 aa, synthetic construct
VEALYLVCGEEG